(19) United States
(12) Patent Application Publication
(10) Pub. No.: US 2025/0368960 A1
(43) Pub. Date: Dec. 4, 2025

(54) THREE-LAYER STRUCTURE INCLUDING EXTRA-CELLULAR MATRIX, SHEET-SHAPED CELL CULTURE, AND BIODEGRADABLE GEL

(71) Applicant: TERUMO KABUSHIKI KAISHA, Tokyo (JP)

(72) Inventors: Fumiya OHASHI, Kanagawa (JP); Ryohei TAKEUCHI, Kanagawa (JP)

(73) Assignee: TERUMO KABUSHIKI KAISHA, Tokyo (JP)

(21) Appl. No.: 19/299,909

(22) Filed: Aug. 14, 2025

Related U.S. Application Data
(62) Division of application No. 17/514,619, filed on Oct. 29, 2021.
Application No. 17/514,619 is a continuation of application No. PCT/JP2020/019847, filed on May 20, 2020.

(30) Foreign Application Priority Data
May 20, 2019 (JP) 2019-094836

(51) Int. Cl. C12N 5/077; B05B 3/12; B05D 1/02

(52) U.S. Cl. CPC C12N 5/0658; B05B 3/12; B05D 1/02; C12N 2513/00; C12N 2533/56; C12N 2533/90; C12N 2539/10

(57) ABSTRACT

A three-layer structure is comprised of an extra-cellular matrix, a sheet-shaped cell culture, and a biodegradable gel layer (single layer). By virtue of this three-layer structure, breakage of the sheet-shaped cell culture due to high spray pressure is reduced. The fibrin gel may be formed on one surface of the sheet-shaped cell culture by spraying of a thrombin liquid. A production method may involve culturing cells on a temperature-responsive cell culture substrate, dripping fibrinogen onto a sheet-shaped cell culture having an extra-cellular matrix on a lower surface thereof, further spraying a thrombin liquid onto the sheet-shaped cell culture from an oblique direction, whereby the result is a three-layer structure having a fibrin gel layer only on one surface of the sheet-shaped cell culture and having an extra-cellular matrix layer on the opposite surface.

### CROSS-REFERENCES TO RELATED APPLICATIONS

This application is a divisional of U.S. patent application Ser. No. 17/514,619 filed on Oct. 29, 2021, which is a continuation of International Patent Application No. PCT/JP2020/019847 filed on May 20, 2020, which claims priority from Japanese Patent Application No. JP2019-094836 filed on May 20, 2019, the entire content of all three of which is incorporated herein by reference.

### TECHNICAL FIELD

The present disclosure relates to a three-layer structure including an extra- cellular matrix, a sheet-shaped cell culture, and a biodegradable gel in this order, a method for producing the structure, and an apparatus used in the production method.

### BACKGROUND DISCUSSION

In recent years, attempts have been made to transplant various cells for repairing damaged tissues and the like. For example, in order to repair myocardial tissues damaged by an ischemic heart disease such as angina or myocardial infarction, use of fetal cardiomyocytes, skeletal myoblast cells, mesenchymal stem cells, cardiac stem cells, ES cells, and the like has been attempted (Haraguchi et al., Stem Cells Transl Med. 2012 February; 1 (2): 136-41).

As an example of such an attempt, a sheet-shaped cell culture in which cells are formed into a sheet shape has been developed. In addition, for application of such a cell culture, for example, a method for detaching cells while retaining an adhesive protein secreted by cells such as an extra-cellular matrix by using a temperature-responsive cell culture substrate has been developed (Japanese Patent No. 4422540). Furthermore, since such a sheet-shaped cell culture is generally fragile, a method for producing a laminate of the sheet-shaped cell culture and a fibrin gel has been attempted in order to solve such a problem (Japanese Patent No. 6495603).

### SUMMARY

However, with regard to the laminate of a sheet-shaped cell culture and a fibrin gel described in JP 6495603 B2, it has been found that when a liquid containing thrombin is sprayed in preparation of the laminate, the sheet-shaped cell culture is damaged by spraying, and a fibrin gel is sometimes generated on both surfaces of the sheet-shaped cell culture to generate a laminate with reduced operability. Therefore, there is a desire for a laminate having higher operability without these problems.

In view of the above, the present inventors have made intensive studies and produced a sheet-shaped cell culture by culturing cells on a cell culture substrate containing a stimulus-responsive material in a structure of a biodegradable gel and a sheet-shaped cell culture. Furthermore, the present inventors have found that when a liquid containing thrombin is sprayed in a case where the biodegradable gel is a fibrin gel, by spraying the liquid at a certain angle with respect to the sheet-shaped cell culture rather than spraying the liquid perpendicularly to the sheet-shaped cell culture, a risk of breakage of the sheet-shaped cell culture due to a spray pressure is reduced, a force of pushing out thrombin and/or fibrinogen on the sheet-shaped cell culture is reduced by such spraying as compared to a force when the liquid is sprayed perpendicularly, and a structure having a fibrin gel layer only on one surface of the sheet-shaped cell culture and having an extra-cellular matrix layer on the opposite surface is provided.
[1] According to one aspect of the disclosure here, a three-layer structure includes an extra-cellular matrix, a sheet-shaped cell culture, and a biodegradable gel in this order.
[2] The structure according to [1], in which the biodegradable gel is a fibrin gel.
[3] A method for producing a three-layer structure including an extra-cellular matrix, a sheet-shaped cell culture, and a fibrin gel in this order, the method including:
   (i) culturing cells on a cell culture substrate containing a stimulus-responsive material to produce a sheet-shaped cell culture having an extra-cellular matrix layer formed on a lower surface thereof;
   (ii) dripping a liquid containing fibrinogen onto an upper surface of the sheet-shaped cell culture;
   (iii) spraying a liquid containing thrombin onto the sheet-shaped cell culture from an oblique direction; and
   (iv) forming a fibrin gel layer on an upper surface of the sheet-shaped cell culture by a reaction between the fibrinogen and the thrombin.
[4] The method according to [3], further including rotating the sheet-shaped cell culture in step (iii).
[5] An apparatus used in the method according to [3] or [4], the apparatus including:
   a spray nozzle for spraying a liquid containing thrombin to form the fibrin gel layer; and
   a stand that supports the spray nozzle and is configured to adjust the spray nozzle to position the spray nozzle at an angle that is oblique in relation to the sheet-shaped cell culture to spray the liquid containing thrombin onto the sheet-shaped cell culture from an oblique direction.
[6] The apparatus according to [5], further including a turntable.
[7] A stand used in the apparatus according to [5] or [6], the stand including a prop and a holder.

A three-layer structure like that disclosed here reduces a risk of breakage of a sheet-shaped cell culture due to a spray pressure during thrombin spraying, has a fibrin gel only on one surface of the sheet-shaped cell culture, and has an extra- cellular matrix layer on the opposite side. Therefore, an adhesive property to a transplantation site is improved when the sheet-shaped cell culture is used for transplantation. That is, a better sheet-shaped cell culture having excellent operability can be provided to a clinical site.

Another aspect involves an apparatus for producing a fibrin gel layer on an upper surface of a sheet-shaped cell culture, which sheet-shaped cell culture has a lower surface on which is located an extra-cellular matrix layer. The apparatus comprises: a turntable having a surface for receiving and supporting a sheet-shaped cell culture, with the turntable being rotatable to rotate the sheet-shaped cell culture supported on the surface of the turntable; a prop shaft held by a shaft holder and extending upwardly from the shaft holder; a spray nozzle holder mounted on an upper portion of the prop; and a spray nozzle for spraying a liquid containing thrombin to form the fibrin gel layer on the upper surface of the sheet-shaped cell culture supported on the surface of the rotatable turntable. The spray nozzle is mounted on the spray nozzle holder at a downwardly angled orientation in which the spray nozzle is at an angle that is oblique in relation to the surface of the rotatable turntable to spray the liquid containing thrombin onto the sheet-shaped cell culture from an oblique direction.

### BRIEF DESCRIPTION OF THE DRAWINGS

FIGS. 1A to 1C illustrate schematic views of a stand 1. FIG. 1A illustrates a side view of the stand 1. FIG. 1B illustrates a front view of the stand 1. FIG. 1C illustrates a plan view of the stand 1 from which holder 7 and prop 3 are removed.

FIGS. 2A to 2D are enlarged views of the holder 7 portion of the stand 1. FIG. 2A illustrates a rear view of the holder 7. FIG. 2B illustrates a plan view of the holder 7. FIG. 2C illustrates a side view of the holder 7. FIG. 2D illustrates a front view of the holder 7.

FIG. 3 illustrates a schematic view of the stand 1 in combination with a turntable 9.

FIG. 4 illustrates a cross-sectional view of a structure produced by a method disclosed herein.

FIG. 5 illustrates a cross-sectional view of a structure produced by dripping a thrombin liquid and a fibrinogen liquid in this order as a comparison.

### DETAILED DESCRIPTION

Set forth below with reference to the accompanying drawings is a detailed description of embodiments of a three-layer structure, a method for producing the three-layer structure, and an apparatus used in the production method representing examples of the inventive three-layer structure, production method and apparatus disclosed here.

One aspect of the disclosure here relates to a three-layer structure including an extra-cellular matrix, a sheet-shaped cell culture, and a biodegradable gel in this order.

In general, the “extra-cellular matrix” is a component secreted from cells and containing water, a protein, a polysaccharide, and the like. Examples thereof include, but are not limited to, collagen, fibronectin, proteoglycan, laminin, hyaluronic acid, tenascin, entactin, elastin, and fibrillin. The extra-cellular matrix serves as a physical scaffold for cells, and also plays an important role in morphogenesis, differentiation, and homeostasis of tissues.

The extra-cellular matrix functions as a physical scaffold, and the extra-cellular matrix layer of the structure thereby contributes to adhesion of the sheet-shaped cell culture to a transplantation site. With such an effect, when the sheet-shaped cell culture is transplanted, a step of adhesion of the sheet-shaped cell culture to a transplantation site, such as suturing, can be simplified, and operability in clinical application is improved. The extra-cellular matrix is a cell-derived component, and therefore there is no disadvantage in using the extra-cellular matrix for transplantation together with the structure. The thickness of a layer of the extra-cellular matrix can be from 1 nm to 100 nm, and preferably from 1 nm to 50 nm.

The term “sheet-shaped cell culture” refers to an object obtained by connecting cells to each other to form a sheet. The cells may be directly connected to each other by a cell adhesion factor or the like, or may be indirectly connected to each other via an extra-cellular matrix or the like. The sheet-shaped cell culture may be formed of a single layer or a multilayer of two layers, three layers, four layers, or more, or may have a three-dimensional structure having no layered structure.

The cells constituting the sheet-shaped cell culture are not particularly limited as long as the cells can form the sheet-shaped cell culture, and examples thereof include an adherent cell (adhesive cell). Examples of the adherent cell include, but are not limited to, an adherent somatic cell (for example, a cardiomyocyte, a fibroblast cell, an epithelial cell, an endothelial cell, a hepatocyte, a pancreatic cell, a kidney cell, an adrenal cell, a periodontal membrane, a gingival cell, a bone membrane cell, a skin cell, a synovial cell, or a chondrocyte) and a stem cell (for example, a tissue stem cell such as a myoblast or a cardiac stem cell, an embryonic stem cell (ES cell), an induced pluripotent stem cell (iPS cell), an embryonic germ cell (EG cell), or a mesenchymal stem cell). The somatic cell may be differentiated from a stem cell, particularly an iPS cell.

The cells constituting the sheet-shaped cell culture can be derived from any organism that is subjected to treatment with the sheet-shaped cell culture, and examples of such an organism include, but are not limited to, humans, primates, dogs, cats, pigs, horses, goats, sheep, rodents (for example, mice, rats, hamsters, or guinea pigs), and rabbits. The sheet-shaped cell culture may be constituted by only one kind of cells, but two or more kinds of cells can also be used. In a preferred embodiment, when the sheet-shaped cell culture is constituted by two or more kinds of cells, the most preferred ratio (purity) is 60% or more, preferably 70% or more, and more preferably 75% or more at an end of production of the sheet-shaped cell culture.

The sheet-shaped cell culture can be produced by any method known to those skilled in the art. For example, the sheet-shaped cell culture can be prepared by a method including a step of seeding cells on a culture substrate, a step of forming the seeded cells into a sheet, and a step of isolating the formed sheet-shaped cell culture from the culture substrate. Preferably, the culture substrate is a temperature-responsive cell culture substrate. More preferably, cells are cultured on the temperature-responsive cell culture substrate from seeding to isolation of the sheet-shaped cell culture for one hour or more in order to form an extra-cellular matrix.

The term “biodegradable gel” refers to a gel that is degraded in vivo, absorbed into a body, metabolized, and excreted. Examples of the biodegradable gel include, but are not limited to, a fibrin gel and a gel formed by AdSpray (registered trademark) (manufactured by Terumo Corporation). The biodegradable gel preferably refers to a gel that generates viscosity by mixing two kinds of liquids. For example, the fibrin gel is a gel having high strength, formed by action of thrombin on fibrinogen by mixing a liquid containing fibrinogen (hereinafter, referred to as a fibrinogen liquid) and a liquid containing thrombin (hereinafter, referred to as a thrombin liquid).

The biodegradable gel layer firmly adheres to the sheet-shaped cell culture, and is useful for preventing breakage of the sheet-shaped cell culture when the sheet-shaped cell culture is detached from a culture vessel and when the sheet-shaped cell culture is moved in order to be used for transplantation or the like. In addition, since the biodegradable gel can be degraded in vivo, the structure can be used for transplantation with the entire biodegradable gel layer. The thickness of the biodegradable gel layer can be from 5 μm to 1300 μm, and preferably from 50 μm to 300 μm.

In one embodiment, the biodegradable gel has a uniform thickness. Here, the uniform thickness means that a difference between the thickest portion and the thinnest portion is 20% or less. The uniform thickness means that a difference in thickness is preferably 10% or less, and more preferably 5% or less. In one embodiment, the biodegradable gel is not hard. Preferably, the biodegradable gel is soft and viscous.

In one embodiment, the structure is laminated or layered in order of an extra-cellular matrix layer, a sheet-shaped cell culture, and a biodegradable gel layer from the bottom.

Another aspect of the disclosure here relates to a method for producing a three-layer structure including an extra-cellular matrix, a sheet-shaped cell culture, and a fibrin gel in this order, the method including: a step of culturing cells on a cell culture substrate containing a stimulus-responsive material to produce a sheet-shaped cell culture having an extra-cellular matrix layer formed on a lower surface thereof; a step of dripping a fibrinogen liquid onto an upper surface of the sheet-shaped cell culture; a step of spraying a thrombin liquid onto the sheet-shaped cell culture from an oblique direction; and a step of forming a fibrin gel layer on an upper surface of the sheet-shaped cell culture by a reaction between the fibrinogen and the thrombin.

The term “cell culture substrate containing a stimulus-responsive material” refers to a substrate whose physical properties change in response to stimulation caused by, for example, temperature, pH, or light, and which is suitable for cell culture. Preferably, the cell culture substrate containing a stimulus-responsive material refers to a temperature-responsive cell culture substrate. More preferably, the temperature-responsive cell culture substrate refers to a substrate exhibiting a cell adhesion property at a temperature at which cells are cultured and exhibiting a cell non-adhesion property at another temperature. In prior art, when a sheet-shaped cell culture is detached from a culture vessel, it is necessary to perform trypsin treatment or the like, and an extra-cellular matrix is damaged by such treatment. However, when cells are cultured on a cell culture substrate containing a stimulus- responsive material including a temperature-responsive cell culture substrate to produce a sheet-shaped cell culture, such treatment at the time of detachment is unnecessary, and the sheet-shaped cell culture can be detached while the extra-cellular matrix is included. As the culture vessel including the cell culture substrate containing a stimulus-responsive material, any known culture vessel can be used. For example, for the temperature-responsive cell culture substrate, Upcell (registered trademark) of CellSeed Co., Ltd. or Cepallet (registered trademark) of DIC Corporation, which are commercially available, can be used.

The fibrinogen liquid is not particularly limited as long as the fibrinogen liquid can react with thrombin to form a fibrin gel, and examples thereof include a liquid containing fibrinogen at a concentration of 1 mg/mL to 500 mg/mL, 5 mg/mL to 400 mg/mL, 10 mg/mL to 250 mg/mL, 20 mg/mL to 150 mg/mL, 40 mg/mL to 100 mg/mL, or 50 mg/mL to 90 mg/mL. A solvent of the fibrinogen liquid is typically water. The fibrinogen liquid may contain, in addition to fibrinogen, a component such as factor XIII, aprotinin, serum albumin, glycine, L-arginine hydrochloride, L-isoleucine, sodium L-glutamate, D-mannitol, sodium citrate hydrate, or sodium chloride. The fibrinogen liquid is commercially available or can be produced based on a known method.

Examples of the commercially available fibrinogen liquid include, but are not limited to, a solution obtained by dissolving the content (fibrinogen lyophilized powder) of Vial 1 of tissue adhesive BOLHEAL (registered trademark) (manufactured by Teijin Pharma Ltd.) in the content (fibrinogen solution) of Vial 2, and a solution obtained by dissolving the content (fibrinogen powder) of Vial 1 of tissue adhesive Beriplast P Combi-Set (registered trademark) (manufactured by CSL Behring) in the content (aprotinin liquid) of Vial 2.

The fibrinogen liquid can be dripped by any known method, for example, using a syringe or a pipette. As the syringe, for example, a needle-less syringe having a volume of 0.5 mL to 5 mL, a needle-equipped syringe (for example, a syringe equipped with a needle of 18 G to 27 G), the two-component mixing set of the preparation set attached to tissue adhesive BOLHEAL (registered trademark) (equipped with an application nozzle about 1 mm in inner diameter, manufactured by Nipro Corporation), or the two-component mixing set of the preparation set of Beriplast (equipped with an application nozzle, manufactured by Nipro Corporation) can be used. The dripping amount of the fibrinogen liquid is not particularly limited as long as the fibrinogen liquid can coat an upper surface of the sheet-shaped cell culture, and may be, for example, about 6 μL/cm² to about 70 μL/cm², about 9 μL/cm² to about 50 μL/cm², about 12 μL/cm² to about 45 μL/cm², about 15 μL/cm² to about 40 μL/cm², or about 18 μL/cm² to about 32 μL/cm^{2.} Examples of the dripping amount of the fibrinogen liquid include, but are not limited to, about 100 μL to about 1000 μL, about 150 μL to about 800 μL, about 200 μL to about 700 μL, about 250 μL to about 600 μL, and about 300 μL to about 500 μL with respect to the sheet-shaped cell culture having a diameter of 45 mm. The particle diameter of a droplet at the time of dripping the fibrinogen liquid is not particularly limited, but may be, for example, in a range in which the diameter of a droplet adhering to the sheet-shaped cell culture after dripping is about 0.2 cm to about 2.0 cm. The weight of the droplet may be, for example, but is not limited to, in a range of about 10 mg to about 100 mg, in a range of about 15 mg to about 50 mg, or in a range of about 20 mg to about 30 mg. The particle size and weight of the droplet can be appropriately adjusted depending on whether or not a needle is attached to a syringe, the number of gauges of a needle to be attached, or the shape of a needle tip.

The thrombin liquid is not particularly limited as long as the thrombin liquid can react with fibrinogen to form a fibrin gel, and examples thereof include a liquid containing thrombin at a concentration of 1 unit/mL to 10,000 units/mL, 10 units/mL to 5000 units/mL, 25 units/mL to 2500 units/mL, 50 units/mL to 1000 units/mL, 100 units/mL to 500 units/mL, or 250 units/mL to 300 units/mL. A solvent of the thrombin liquid is typically water. The thrombin liquid may contain, in addition to thrombin, a component such as sodium citrate hydrate or sodium chloride. The thrombin liquid is commercially available or can be produced based on a known method. Examples of the commercially available thrombin liquid include, but are not limited to, a solution obtained by dissolving the content (thrombin lyophilized powder) of Vial 3 of tissue adhesive BOLHEAL (registered trademark) (manufactured by Teijin Pharma Ltd.) in the content (thrombin solution) of Vial 4, and a solution obtained by dissolving the content (thrombin powder) of Vial 3 of tissue adhesive Beriplast P Combi-Set (registered trademark) (manufactured by CSL Behring) in the content (calcium chloride liquid) of Vial 4.

The thrombin liquid can be sprayed by any known method, for example, using a spray. Examples of the spray include, but are not limited to, a BOLHEAL (registered trademark) spray set (manufactured by Akita Sumitomo Bakelite Co., Ltd.) and a spray obtained by attaching a spray chip to the two-liquid mixing set attached to tissue adhesive Beriplast P Combi-Set (registered trademark) (manufactured by CSL Behring). The spray amount of the thrombin liquid is not particularly limited as long as the thrombin liquid can cover an upper surface of the sheet-shaped cell culture, and may be, for example, about 3 μL/cm² to about 70 L/cm², about 5 μL/cm² to about 50 μL/cm², about 6 μL/cm² to about 45 μL/cm², about 12 μL/cm² to about 40 μL/cm², or about 18 μL/cm² to about 32 μL/cm² in terms of an estimated adhesion amount to the sheet-shaped cell culture. Non-limiting examples of the spray amount of the thrombin liquid include about 50 μL to about 1000 μL, about 80 μL to about 800 μL, about 100 μL to about 700 μL, about 200 μL to about 600 μL, and about 300 μL to about 500 μL in terms of an estimated adhesion amount to the sheet-shaped cell culture having a diameter of 45 mm. The estimated adhesion amount to the sheet-shaped cell culture is calculated by measuring the weight of a liquid adhering to a predetermined region (for example, within a circle having a diameter of 45 mm) when a predetermined amount of the thrombin liquid is sprayed with a spray, a height, a spray pressure, and a spray angle to be actually sprayed, and dividing the weight by the density of the thrombin liquid (0.999973 g/cm³). Those skilled in the art can determine, based on the description herein, spray conditions that result in a desired estimated adhesion amount of the thrombin liquid without undue experimentation. For example, it has been confirmed that the estimated adhesion amounts with respect to the spray amounts of 300 μL, 500 μL, 600 μL and 900 μL are 100 μL, 180 μL, 300 μL and 450 μL, respectively. Therefore, an approximate curve of estimated adhesion amount (μL)=spray amount (μL)×0.6−88 (μL) is obtained by a least squares method, and the spray amount that results in a desired estimated adhesion amount can be determined based on this approximate curve.

The thrombin liquid is sprayed onto a fibrinogen liquid evenly dripped onto the sheet-shaped cell culture. Here, the thrombin liquid should be sprayed such that the sprayed thrombin liquid and/or the dripped fibrinogen liquid does not reach a lower surface of the sheet-shaped cell culture and the sheet-shaped cell culture is not damaged by spraying. When the sprayed thrombin liquid and/or the fibrinogen liquid reaches a lower surface of the sheet-shaped cell culture, a fibrin gel layer is also formed on the lower surface of the sheet-shaped cell culture. In this case, at the time of transplantation, the fibrin gel layer is present between the sheet-shaped cell culture and a transplantation site. This prevents the sheet-shaped cell culture from coming into contact with the transplantation site, and reduces operability, for example, prevents exchange of substances secreted by cells including an extra-cellular matrix related to cell adhesion.

In general, when a liquid is sprayed using a spray nozzle, the liquid is sprayed straight onto a spray target from a perpendicular direction. However, when a liquid is sprayed onto the sheet-shaped cell culture from a perpendicular direction in this way, the entire spray pressure is applied perpendicularly to the sheet-shaped cell culture, and a risk of damaging the sheet-shaped cell culture is increased. In addition, the fibrinogen liquid that has been dripped and/or the sprayed thrombin liquid can be strongly pushed out away by a reaction caused when the high spray pressure hits the sheet-shaped cell culture. If a distance from the sheet-shaped cell culture is set to be long in order to suppress the spray pressure, the thrombin liquid sprayed from a nozzle spreads more widely and radially. Therefore, the thrombin liquid reaches a lower surface of the sheet-shaped cell culture along an edge of a culture vessel. In addition, the thrombin liquid is sprayed also onto a periphery of the sheet-shaped cell culture, and an extra thrombin liquid to be sprayed is required.

Spraying is performed from an oblique direction. The spray pressure is dispersed in a direction perpendicular to the sheet-shaped cell culture and a direction parallel to the sheet-shaped cell culture by spraying from an oblique direction, and the pressure applied to the sheet-shaped cell culture is smaller than a pressure applied by spraying from the perpendicular direction. Therefore, it is considered that a risk of damaging the sheet-shaped cell culture is also reduced. The thrombin liquid is evenly distributed by the pressure dispersed in a direction parallel to the sheet-shaped cell culture. The pressure dispersed in the horizontal direction with respect to the sheet-shaped cell culture at this time is weaker than the pressure applied or exerted onto the sheet-shaped cell culture when the thrombin liquid is sprayed from a perpendicular direction. In addition, the pressure dispersed in the horizontal direction with respect to the sheet-shaped cell culture prevents fibrinogen that has already reacted with thrombin from being further pushed away, thereby reducing the risk of pushing out the fibrinogen liquid and/or the thrombin liquid from an edge of the sheet-shaped cell culture to form a fibrin gel on a lower surface of the sheet-shaped cell culture, thereby also further reducing the pressure applied to the sheet-shaped cell culture in the perpendicular direction. Therefore, it is considered that a distance does not need to be set to be longer than a distance when the thrombin liquid is sprayed from the perpendicular direction, and the liquid does not travel along an edge of the culture vessel.

Such spraying from an oblique direction is determined by a height and an angle at which the thrombin liquid is sprayed onto the sheet-shaped cell culture. By performing spraying from a spray position of a spray nozzle at a height of about 2 cm to about 15 cm with respect to the sheet-shaped cell culture from a direction inclined by about 15° to about 150° with respect to the sheet-shaped cell culture at a pressure of about 0.005 MPa to about 0.1 MPa, the single layer of fibrin gel can be achieved. Preferably, spraying is performed from a spraying position of a spray nozzle at a height of 7 cm high with respect to the sheet-shaped cell culture from a direction inclined by 45° with respect to the sheet-shaped cell culture.

In a preferred embodiment, the thrombin liquid is sprayed while the sheet-shaped cell culture is rotated. Here, “rotation” refers to rotating the sheet-shaped cell culture around an axis passing through the center of the sheet-shaped cell culture and perpendicular to a plane including the sheet-shaped cell culture. By rotating the sheet-shaped cell culture, the thickness of the fibrin gel layer can be made more uniform. This makes it possible to further reduce unevenness of the thickness of the fibrin gel in the structure, and to prevent a thin portion from being damaged, for example, when the sheet-shaped cell culture is detached from the culture vessel or moved to a transplantation site. The rotation may be performed manually by an operator or may be performed using a tool such as a turntable. In addition, the rotation may be a combination of longitudinal and/or lateral swinging, for example, when the sheet-shaped cell culture is curled. Preferably, the rotation continues to be performed while spraying is performed.

In one embodiment, the step of preparing a fibrin gel can be performed before the sheet-shaped cell culture is detached from the temperature-responsive cell culture substrate. In such an embodiment, a surface of the sheet-shaped cell culture forming the extra-cellular matrix adheres to the temperature-responsive cell culture substrate, and each of the liquids does not enter the surface. Therefore, a risk of forming a fibrin gel on a side of the sheet-shaped cell culture containing the extra-cellular matrix can be reduced. In one embodiment, the method is performed under aseptic conditions in all steps.

A ratio between the fibrinogen liquid and the thrombin liquid applied to the sheet-shaped cell culture is not particularly limited as long as the ratio does not excessively inhibit a transplantation operation of a structure to be obtained. For example, a volume ratio between the dripping amount of the fibrinogen liquid and the estimated adhesion amount of the thrombin liquid is about 5:1 to about 1:3, about 4:1 to about 1:2, about 3:1 to about 1:1.5, about 2.5:1 to about 1:1, about 2:1 to about 1:1, or about 1.5:1 to about 1:1, and preferably about 1:1. A ratio (mg: unit) between fibrinogen and thrombin adhering to the structure is about 8:5 to about 8:75, about 32:25 to about 4:25, about 24:25 to about 16:75, about 4:5 to about 8:25, about 16:25 to about 8:25, or about 12:25 to about 8:25, and preferably about 8:25.

By adjusting the concentrations or amounts of the fibrinogen liquid and the thrombin liquid, the thickness and properties (flexibility, adhesiveness, and the like) of a structure to be obtained can be changed. For example, by increasing the amount of the fibrinogen liquid, the thickness of the structure can be increased. As the volume ratio between the dripping amount of the fibrinogen liquid (80 mg/mL) and the estimated adhesion amount of the thrombin liquid (250 units/mL) is brought closer to 1:1, and as the ratio (mg: unit) between fibrinogen and thrombin adhering to the structure is brought closer to 8:25, the flexibility and adhesiveness of the structure are enhanced, and operability is improved.

The step of forming a fibrin gel layer is not limited and can be performed by, for example, allowing the sheet-shaped cell culture to stand for a certain period of time after the thrombin liquid is sprayed. The standing period is, but is not limited to, for example, about 1 minute to about 60 minutes, about 2 minutes to about 30 minutes, about 3 minutes to about 20 minutes, or about 4 minutes to about 10 minutes, more specifically, for example, about 1 minute, about 2 minutes, about 3 minutes, about 4 minutes, about 5 minutes, about 6 minutes, about 7 minutes, about 8 minutes, about 9 minutes, or about 10 minutes, and preferably about 5 minutes.

Another aspect of the disclosure here relates to an apparatus for spraying a liquid containing thrombin from an oblique direction, the apparatus including a spray nozzle and a stand.

The “spray nozzle” can be any known spray nozzle and examples thereof include, but are not limited to, a BOLHEAL (registered trademark) spray set (manufactured by Akita Sumitomo Bakelite Co., Ltd.) and a spray nozzle obtained by attaching a spray chip to the two-liquid mixing set attached to tissue adhesive Beriplast P Combi-Set (registered trademark) (manufactured by CSL Behring).

The “stand” is used for supporting a spray nozzle for spraying thrombin, and is used for determining a position of the spray nozzle, that is, a spray starting position of a tip of the spray nozzle and an angle of the spray nozzle, and performing spraying at a constant angle to the sheet-shaped cell culture from the position. Here, the angle of the spray nozzle refers to an inclination of the spray nozzle with respect to a spray target, that is, a plane including the sheet-shaped cell culture. In one embodiment, the stand is used after sterilization.

Hereinafter, various embodiments will be described in detail with reference to the drawings. The numerical values in the drawings indicate numerical values in a preferred embodiment, and should not be limited thereto.

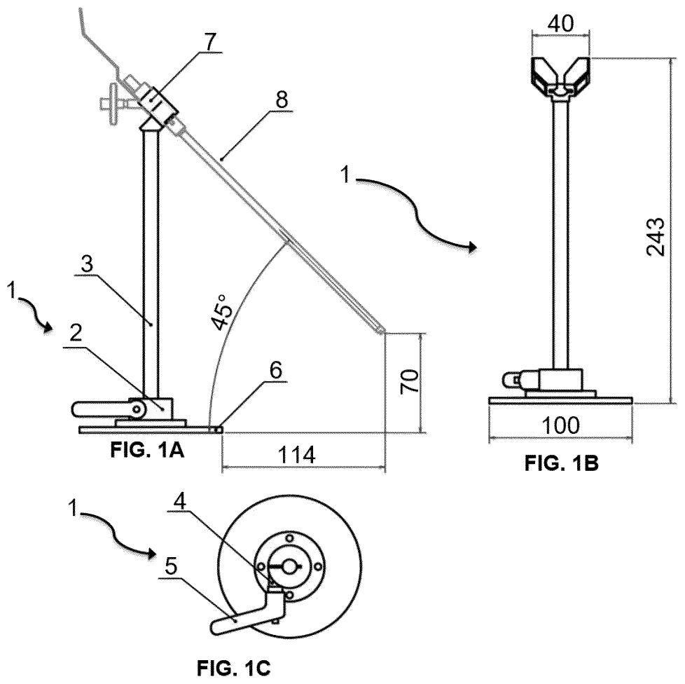

FIG. 1 illustrates a three-sided view of a stand. In FIG. 1, a stand 1 includes a shaft holder 2, a prop 3, a collar 4, a clamp lever 5, a plate 6, and a holder 7. FIG. 1A illustrates a side view of the stand 1 including a spray nozzle 8. The height of the stand 1 can be varied depending on the size of the prop 3, and a spray position of a spray nozzle can be adjusted to a preferable height in the above method for producing a structure. The prop 3 is fixed by the shaft holder 2, and is stably disposed on a work table by the plate 6. The holder 7 is attached to an upper end of the prop 3, and the holder fixes the spray nozzle 8. The angle of the spray nozzle 8 can be varied depending on an attachment angle of the holder 7 with respect to the prop 3, and a spray angle can be adjusted to a preferable angle in the above method for producing a structure. FIG. 1B illustrates a front view of the stand 1. FIG. 1C illustrates a plan view of the stand 1 from which the holder 7 and the prop 3 are removed. In FIG. 1C, the metal collar 4 and the clamp lever 5 are illustrated. In one embodiment, by operating the clamp lever 5, the shaft holder 2 can be loosened or tightened, and the prop 3 can be thereby detached or attached.

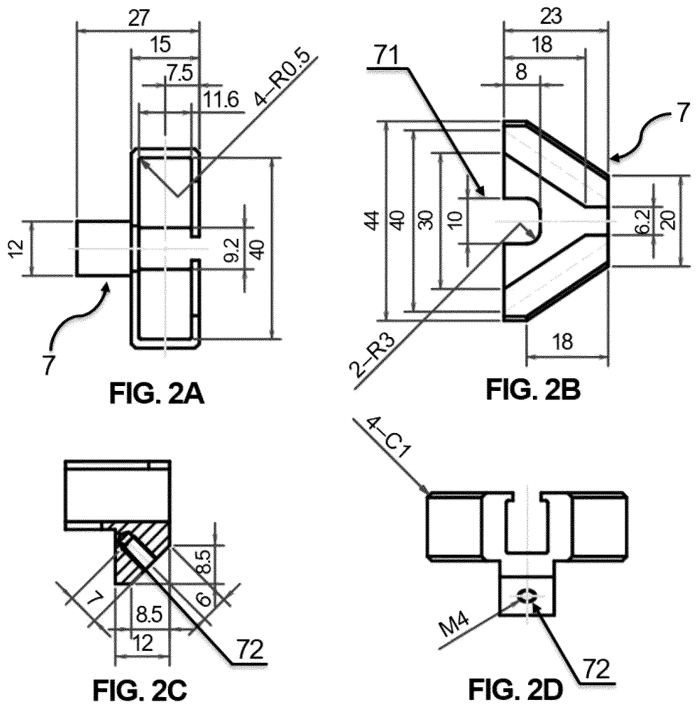

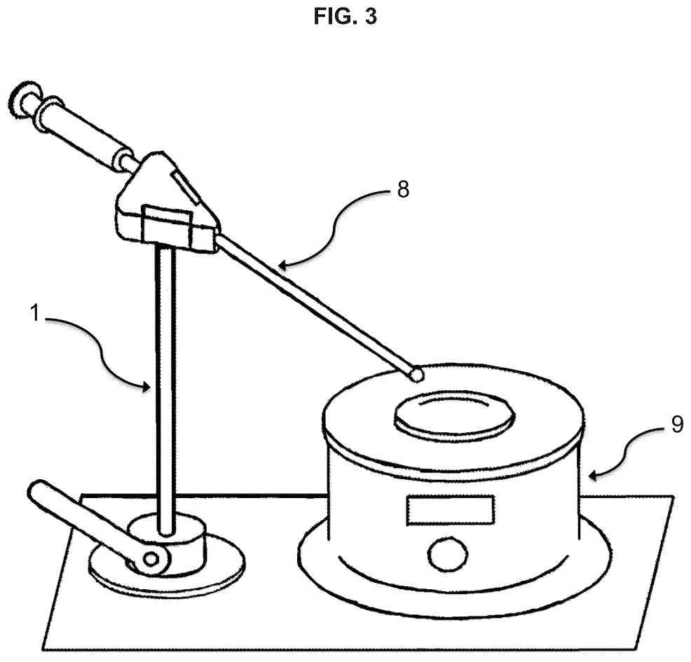

FIG. 2 is an enlarged four-sided view of the holder 7 in the stand 1 illustrated in FIG. 1. As illustrated in FIGS. 2A and 2B, the holder 7 has a suitable shape to which the spray nozzle is fixed. For example, in an embodiment of using a spray tip of a BOLHEAL (registered trademark) spray set (manufactured by Akita Sumitomo Bakelite Co., Ltd.) or tissue adhesive Beriplast P Combi-Set (registered trademark) (manufactured by CSL Behring), the holder 7 has a shape in which the opening size decreases toward a tip. In addition, a slit 71 in the plane view of FIG. 2B makes passage of an air line for supplying gas to the spray nozzle possible when the spray nozzle described above is used. FIGS. 2C and 2D illustrate a side view and a front view of the holder 7, respectively. In each of FIGS. 2C and 2D, an opening 72 connecting the prop 3 is illustrated. An angle of the holder, that is, an angle of the spray nozzle fixed to the holder can be adjusted by an angle of the opening with respect to the holder body.

When the stand 1 is used, the prop 3 and the holder 7 are adjusted in advance so as to have a height and an angle suitable for use in the above method for producing a structure, and in such a state, the spray nozzle 8 is fixed to the holder 7 such that an air line matches the slit portion 71 of the holder 7. By using the stand, an operator can continue to perform spraying under certain conditions such as spray height and angle, and proficiency for performing the spraying work is reduced. In addition, since one hand is available by fixing the spray nozzle, the thrombin liquid can be sprayed while the sheet-shaped cell culture is rotated even in an operation by one operator.

A material constituting the stand 1 is not particularly limited, but is preferably a sterilizable material. Examples of the sterilizable material include, but are not limited to, stainless steel, polypropylene, polyvinylidene fluoride, and a tetrafluoroethylene-ethylene copolymer resin. Preferably, the sterilizable material is stainless steel.

In one embodiment, the apparatus further includes a turntable.

The term “turntable” refers to a tool including a top plate on which a culture vessel containing the sheet-shaped cell culture can be placed, and capable of rotating the top plate at a constant angular velocity. In one embodiment, the turntable is an electric turntable that continues to turn automatically when a setting is input thereto.

FIG. 3 is a schematic view of the apparatus including the turntable. For ease of explanation, the size of each of members and the positional relationship between the members in FIG. 3 are emphasized as appropriate, and the size of each of members and the positional relationship between the members illustrated in FIG. 3 do not indicate actual ones.

As illustrated in FIG. 3, in the apparatus, a liquid is sprayed from the spray nozzle 8 fixed by the stand 1 onto a sample rotating on the turntable 9. According to such an embodiment, since rotation of the sheet-shaped cell culture and support of the spray nozzle are performed at the time of spraying the thrombin liquid in the above method for producing a structure, an operator can perform the spraying step only by pushing a syringe of the thrombin liquid through the spray nozzle 8. Regarding a positional relationship between the stand 1 and the turntable 9, it is desirable that the center of a sample on the turntable 9 is located at a position where a tip of the spray nozzle 8 supported by the stand 1 is extended. The stand 1 and the turntable 9 may be physically connected to each other or separated from each other.

### EXAMPLES

### Example 1. Production of Structure

Skeletal myoblast cells (CD56 positive) cryopreserved in a preservation liquid for cell freezing (MCDB culture medium containing 10% DMSO) were thawed at 37° C., and washed twice using a physiological buffer containing 0.5% serum albumin. 6.0×10⁷ washed cells were suspended in a DMEM culture medium containing 20% human blood serum (10 mL), and seeded on a 10-cm-diameter cell culture dish (UpCell (registered trademark) 10-cm dish, CS3005, manufactured by CellSeed Inc.). After seeding, the cells were cultured in an incubator set at 37° C. and 5% CO₂ (BNA-121D, manufactured by Espec Corp.) for 20 hours. After culturing, the culture dish was taken out from the incubator. After confirming presence of a sheet-shaped cell culture adhering to cover the entire bottom of the culture dish, the culture medium was discarded. Thereafter, the sheet-shaped cell culture was detached from the culture dish by a temperature treatment (allowing to stand at room temperature (20 to 25° C.) for five to 30 minutes) and pipetting. The size of the obtained sheet-shaped cell culture was 47 mm×47 mm.

The culture solution in the culture dish was removed, and the sheet-shaped cell culture was shaped as necessary. Thereafter, onto the sheet-shaped cell culture, 500 μL of a fibrinogen liquid (a solution obtained by dissolving the content of Vial 1 (a lyophilized powder of fibrinogen) of tissue adhesive BOLHEAL (registered trademark) (manufactured by Teijin Pharma Ltd.) in the content of Vial 2 (a fibrinogen solution), fibrinogen concentration: 80 mg/mL; the same hereinafter) was dripped using the two-component mixing set of the preparation set attached to tissue adhesive BOLHEAL (registered trademark) (equipped with an application nozzle about 6 cm in length and about 1 mm in inner diameter, manufactured by Nipro Corporation). Subsequently, 800 μL of a thrombin liquid (a solution obtained by dissolving the content of Vial 3 (a lyophilized powder of thrombin) of tissue adhesive BOLHEAL (registered trademark) (manufactured by Teijin Pharma Ltd.) in the content of Vial 4 (a thrombin solution), thrombin concentration: 250 units/mL; the same hereinafter) was sprayed using BOLHEAL (registered trademark) spray set (manufactured by Akita Sumitomo Bakelite Co., Ltd) from a spray nozzle placed about 7 cm away from the cell sheet at a pressure of 0.03 MPa. The sheet-shaped cell culture contracts by being detached from the culture dish and becomes smaller than the bottom of the culture dish. Therefore, it is estimated that out of 800 μL sprayed, about 500 μL of the thrombin liquid adhered onto the sheet-shaped cell culture. The estimated adhesion amount of the thrombin liquid was calculated as follows. A predetermined amount of thrombin liquid (in this case, 800 μL) was actually sprayed using a predetermined spray (in the case of this example, BOLHEAL (registered trademark) spray set) at spray pressure (in the case of this example, 0.03 MPa) at height (in the case of this example, about 7 cm) at spray angle (in the case of this example,) 45°, and the weight of the thrombin liquid adhering to a predetermined region (in the case of this example, within a circle having a diameter of about 45 mm) was measured with an electronic balance and divided by the density of the thrombin liquid.

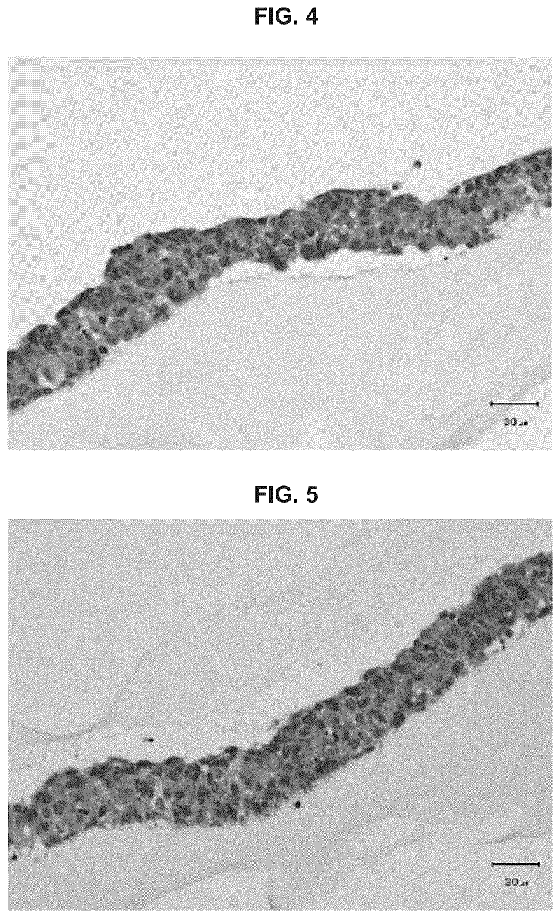

A fibrin gel is formed by a reaction in dripping of the fibrinogen liquid and following spraying of the thrombin liquid. After the culture dish was allowed to stand for about five minutes, 24 mL of Hanks' balanced salt solution (HBSS (+), Cat No. 14025, manufactured by Life Technologies Corporation; the same hereinafter) was added to the culture dish and immediately removed, thereby washing the culture dish containing the sheet-shaped cell culture. This makes it possible to remove unreacted fibrinogen liquid and thrombin liquid. Subsequently, 24 mL of Hanks' balanced salt solution was added again to the culture dish, and the culture dish was allowed to stand for about 15 minutes. Thereafter, the solution in the culture dish was removed, the fibrin gel solidified other than on the sheet-shaped cell culture was trimmed with a scalpel, and a structure of the fibrin gel and the sheet-shaped cell culture was isolated (Structure 1). When the cross section of the isolated structure was examined, it was confirmed that the fibrin gel was formed only on an upper surface of the sheet-shaped cell culture (upside down in FIG. 4), and the thickness of the gel was uniform without unevenness (FIG. 4).

For comparison, an attempt was made to produce a structure of a fibrin gel and a sheet-shaped cell culture (Comparative Structure 1) by a method for dripping a thrombin liquid and a fibrinogen liquid in this order.

Similarly to Structure 1, a sheet-shaped cell culture was formed and detached from a culture dish. The culture solution in the culture dish was removed, and the sheet-shaped cell culture was shaped as necessary. Thereafter, 800 μL of a thrombin liquid and 800 μL of a fibrinogen liquid were sequentially dripped onto the sheet-shaped cell culture using the two-component mixing set of the preparation set attached to tissue adhesive BOLHEAL (registered trademark) (equipped with an application nozzle about 6 cm in length and about 1 mm in inner diameter, manufactured by Nipro Corporation). After the culture dish was allowed to stand for about five minutes, 24 mL of Hanks' balanced salt solution was added to the culture dish and immediately removed, thereby washing the culture dish containing the sheet-shaped cell culture. Subsequently, 24 mL of Hanks' balanced salt solution was added again to the culture dish and allowed to stand for about 15 minutes, and then the solution in the culture dish was removed. When the cross section was confirmed, it was confirmed that the fibrin gel was formed not only on an upper surface of the sheet-shaped cell culture but also on a lower surface thereof (upside down in FIG. 5). Furthermore, the fibrin gel layer did not have a uniform gel thickness, and operability was deteriorated (FIG. 5).

Similarly, for comparison, an attempt was made to produce a structure of a fibrin gel and a sheet-shaped cell culture by a method for dripping a fibrinogen liquid and a thrombin liquid in this order (Comparative Structure 2), a structure obtained by spraying both a fibrinogen liquid and a thrombin liquid (Comparative Structure 3), and a structure obtained by dripping a thrombin liquid and then spraying a fibrinogen liquid (Comparative Structure 4). The production was performed by the same method as the method for producing Comparative Structure 1 except for dripping and/or spraying the liquids and order thereof.

Comparative Structure 2 was a thick structure having a thickness of more than 1300 μm, was formed into a hard gel, and was not suitable for a transplantation operation. In Comparative Structure 3, the fibrin gel was detached from the sheet-shaped cell culture, and a structure was not formed. In Comparative Structure 4, the fibrin gel layer was formed in spots, a back side of the layer was not solidified, and a structure was not formed.

### Example 2. Evaluation of Structure

By the same procedure as in the production of Structure 1, Structure 2 was produced except that the dripping amount of the fibrinogen liquid was 300 μL, and the spraying amount of the thrombin liquid was about 600 μL (estimated adhesion amount: about 300 μL), Structure 3 was produced except that the dripping amount of the fibrinogen liquid was 300 μL, and the spraying amount of the thrombin liquid was about 900 μL (estimated adhesion amount: about 450 μL), Structure 4 was produced except that the dripping amount of the fibrinogen liquid was 300 μL, and the spraying amount of the thrombin liquid was about 300 μL (estimated adhesion amount: about 100 μL), and Structure 5 was produced except that the dripping amount of the fibrinogen liquid was 500 μL, and the spraying amount of the thrombin liquid was about 500 μL (estimated adhesion amount: about 180 μL).

Structure 1 obtained in Example 1 and Structures 2 to 5 were evaluated in terms of size, weight, strength, thickness, and properties. Size was measured with a ruler, weight was measured with an electronic non-automatic scale (AT201, manufactured by Mettler-Toledo), and thickness was measured with a dial thickness gauge (SM-124, manufactured by Teclock Corporation). Strength was measured as follows. First, a structure extended in a liquid was scooped up with an intestinal spatula made of stainless steel (45 mm in width; the same hereinafter) and placed out of the liquid with the structure adhering to a surface of the intestinal spatula. A suture equipped with a needle (6-0 proline) was caused to pass through the structure from a lower surface to an upper surface. Both ends of the thread were tied together to form a ring, which was then connected to a gauge (a general-purpose digital force gage, FGC-1B, manufactured by Nidec-Shimpo Corporation). The thread locked to the structure was horizontally pulled through the gauge, and a maximum load before the structure broke (tensile breaking load) was measured. Strength was measured at three different points on a structure (n=3). Properties were qualitatively evaluated based on a state observed during an operation of transferring the structure to a side surface of a bottle imitating the heart.

TABLE 1
Evaluation results of Structures 1 to 5

| | Structure | Structure | Structure | Structure | Structure |
|---|---|---|---|---|---|
| | 1 | 2 | 3 | 4 | 5 |
| FN:TN (μL) * | 500:500 | 300:300 | 300:450 | 300:100 | 500:180 |
| FN:TN ratio** | 1:1 | 1:1 | 1:1.5 | 3:1 | 3:1 |
| Size of sheet- | 47 × 47 | — | — | — | 45 × 46 |
| shaped cell | | | | | |
| culture (mm) | | | | | |
| Size (mm) | 45 × 42 | 43 × 42 | 50 × 46 | 42 × 45 | 45 × 40 |
| Weight (g) | 1.92995 | 1.26149 | 1.24936 | 0.98233 | 1.5484 |
| Strength (N) | 0.047 | 0.020 | 0.023 | 0.033 | 0.043 |
| (n = 3) | | | | | |
| Thickness (mm) | 1.250 | 0.467 | 0.433 | 0.615 | 1.337 |

| | | |
|---|---|---|
| Properties | Soft and viscous | Hard |

* FN represents the dripping amount of fibrinogen liquid, and TN represents the estimated adhesion amount of thrombin liquid.
**A ratio between the dripping amount of fibrinogen liquid and the estimated adhesion amount of thrombin liquid

From the results in Table 1, it has become clear that a difference in strength depending on a mixing ratio is not large, but there is a difference in properties such as weight, hardness, and viscosity depending on the mixing ratio. In addition, based on ease of operation in the operation of transferring a laminate structure to a bottle imitating the heart (ease of placement on intestinal spatula, difficulty in falling from intestinal spatula during movement, and ease of transfer from intestinal spatula to bottle) and the state of a structure at the time of operation (whether or not wrinkling, tearing, or the like occurs), a structure having a mixing ratio of 1:1 exhibited the best operability.

By using the three-layer structure disclosed here, it is possible to reduce a risk of breakage when a produced sheet-shaped cell culture is detached from a culture substrate and moved to a transplantation site or the like, and it is possible to simplify a step of adhesion of the sheet-shaped cell culture to the transplantation site. By providing such a sheet-shaped cell culture having high operability, regenerative medicine using the sheet-shaped cell culture can be more widely spread.

The detailed description above describes embodiments of a three-layer structure, a method for producing a three-layer structure, and an apparatus used to perform the production method representing examples of the inventive three-layer structure, production method and apparatus disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A method for producing a three-layer structure including an extra-cellular matrix, a sheet-shaped cell culture, and a fibrin gel in this order, the method comprising:
culturing cells on a cell culture substrate containing a stimulus-responsive material to produce a sheet-shaped cell culture having an extra-cellular matrix layer formed on a lower surface thereof;
dripping a liquid containing fibrinogen onto an upper surface of the sheet-shaped cell culture;
spraying a liquid containing thrombin onto the sheet-shaped cell culture from an oblique direction; and
forming a fibrin gel layer on an upper surface of the sheet-shaped cell culture by a reaction between the fibrinogen and the thrombin liquids.

2. The method according to claim 1, further comprising rotating the sheet-shaped cell culture during the spraying of the liquid containing thrombin onto the sheet-shaped cell culture from an oblique direction.

3. The method according to claim 1, wherein the liquid containing thrombin is sprayed onto the sheet-shaped cell culture from a direction inclined by about 15° to about 150° with respect to the sheet-shaped cell culture.

4. The method according to claim 1, wherein the liquid containing thrombin is sprayed onto the sheet-shaped cell culture at a pressure of about 0.005 MPa to about 0.1 MPa.

5. The method according to claim 1, wherein the liquid containing thrombin is sprayed onto the sheet-shaped cell culture at a height of about 2 cm to about 15 cm above the sheet-shaped cell culture.

6. The method according to claim 1, wherein an amount of the thrombin liquid sprayed onto the sheet-shaped cell culture is about 3 μL/cm² to about 70 μL/cm².

7. The method according to claim 1, wherein an amount of the fibrinogen liquid dripped onto the sheet-shaped cell culture is about 6 μL/cm² to about 70 μL/cm².

8. The method according to claim 1, wherein the spraying of the liquid containing thrombin onto the sheet-shaped cell culture from the oblique direction includes spraying the liquid containing thrombin from a spray nozzle that is mounted on an upstanding prop shaft.

9. An apparatus for producing a fibrin gel layer on an upper surface of a sheet-shaped cell culture, which sheet-shaped cell culture has a lower surface on which is located an extra-cellular matrix layer, the apparatus comprising:
a turntable having a surface for receiving and supporting a sheet-shaped cell culture, the turntable being rotatable to rotate the sheet-shaped cell culture supported on the surface of the turntable;
a prop shaft held by a shaft holder and extending upwardly from the shaft holder;
a spray nozzle holder mounted on an upper portion of the prop;
a spray nozzle for spraying a liquid containing thrombin to form the fibrin gel layer on the upper surface of the sheet-shaped cell culture supported on the surface of the rotatable turntable, the spray nozzle being mounted on the spray nozzle holder at a downwardly angled orientation in which the spray nozzle is at an angle that is oblique in relation to the surface of the rotatable turntable to spray the liquid containing thrombin onto the sheet-shaped cell culture from an oblique direction.

10. The apparatus according to claim 9, wherein the shaft holder includes a clamp that is operable to loosen and tighten the shaft holder.

11. The apparatus according to claim 9, wherein the shaft holder is is mounted on a plate.

12. The apparatus according to claim 9, wherein spray nozzle holder includes a slit for receiving a gas line for supplying gas to the spray nozzle held in the spray nozzle holder.

13. The apparatus according to claim 9, wherein spray nozzle holder includes an opening in which is received the prop shaft.

14. An apparatus for producing a fibrin gel layer on an upper surface of a sheet-shaped cell culture, which sheet-shaped cell culture has a lower surface on which is located an extra-cellular matrix layer, the apparatus comprising:
a spray nozzle for spraying a liquid containing thrombin to form the fibrin gel layer; and
a stand that supports the spray nozzle and is configured to adjust the spray nozzle to position the spray nozzle at an angle that is oblique in relation to the sheet-shaped cell culture to spray the liquid containing thrombin onto the sheet-shaped cell culture from an oblique direction.

15. The apparatus according to claim 14, further comprising a turntable.

16. The apparatus according to claim 14, wherein the stand further comprises a prop and a holder.

17. The apparatus according to claim 16, wherein the stand further comprises a shaft holder, a prop, a collar, a clamp lever, and a plate.

18. The apparatus according to claim 14, wherein the stand is comprised of a sterilizable material.

19. The apparatus according to claim 14, wherein the spray nozzle is obtained by attaching a spray chip to a two-liquid mixing set attached to a tissue adhesive.
